(19) United States
(12) Patent Application Publication
(10) Pub. No.: US 2026/0240511 A1
(43) Pub. Date: Aug. 20, 2026

(54) METHODS FOR ANGIOGRAPHY

(71) Applicant: William E. BUTLER, Bristol, RI (US)

(72) Inventor: William E. BUTLER, Bristol, RI (US)

(21) Appl. No.: 19/460,991

(22) Filed: Jan. 27, 2026

Related U.S. Application Data
(63) Continuation of application No. 18/795,413, filed on Aug. 6, 2024, now Pat. No. 12,551,178.
Application No. 18/795,413 is a continuation of application No. 16/784,073, filed on Feb. 6, 2020, now Pat. No. 12,076,174.
(60) Provisional application No. 62/801,780, filed on Feb. 6, 2019.

(51) Int. Cl. A61B 6/00; A61B 6/50

(52) U.S. Cl. CPC A61B 6/481; A61B 6/503; A61B 6/504; A61B 6/5205; A61B 6/5217; A61B 6/5258

(57) ABSTRACT

An angiogram is a study of blood vessels where an angiographic chemical contrast agent is injected while a sequence of images (typically x-rays) are obtained. The contrast pattern on the sequence of images provides information about the vascular anatomy and physiology. The discovery that contrast in blood vessels varies at cardiac frequency in magnitude and phase, which may be visualized as a spatiotemporal reconstruction of cardiac frequency angiographic phenomena, enables a set of processes for increasing the signal to noise ratio or equivalently the informational content of an angiogram. In this invention, the organization of cardiac frequency magnitude and phase enables equivalent information on anatomy and physiology to be obtained with less dose of injected chemical contrast agent, less x-ray dose, and/or less navigation of the injecting catheter within blood vessels. The cardiac frequency magnitude and phase is organized so that the arterial and venous subsystems of circulation have coherence at cardiac frequency. This enables processes for diagnosing deficits of circulation that involve alterations in the transit of blood from the arterial to the venous subsystems of circulation. Furthermore, the discovery of cardiac frequency magnitude and phase organization enables the design and manufacture of lighter and more portable angiography equipment.

### CROSS-REFERENCE TO RELATED APPLICATIONS

This application is a continuation of U.S. patent application Ser. No. 18/795,413, filed on Aug. 6, 2024, which is a continuation of U.S. patent application Ser. No. 16/784,073, filed on Feb. 6, 2020, now U.S. Pat. No. 12,076,174, which claims priority to U.S. Provisional Application No. 62/801,780, filed Feb. 6, 2019, the contents of which are hereby incorporated by reference herein in their entireties.

### FIELD OF THE INVENTION

Improved methods for angiography are provided. Specifically, methods of obtaining angiographic data are provided that permit use of greatly reduced doses of contrast agent and/or x-ray dosage, while maintaining, or improving the signal to noise ratio of the angiogram.

### BACKGROUND OF THE INVENTION

In a conventional catheter angiogram, a catheter is placed into an artery and the catheter tip is advanced into the arterial region of interest. A chemical contrast agent is injected and the passage of the contrast to the vascular bed is fluoroscopically imaged and recorded. The contrast agent is opaque to the x-ray, causing a pattern of opacification to appear on the imaging x-ray detector in a sequence of angiographic image frames. Vascular anatomy may be characterized by the opacification pattern in an image in relation to normative patterns of anatomy as recorded in textbooks and other resources. A signal to noise ratio may be measured by comparing (a) the degree of opacification of a contrast-containing vessel upon projection onto the x-ray detector and (b) background, where the background is defined by regions without vascular anatomy containing chemical contrast agent and/or auto-fluorescence.

The anatomy is further characterized by the passage timing properties of the bolus of injected contrast agent. The chemical contrast agent passes through the arterial subsystem of circulation, the capillary subsystem, and then the venous subsystem, with overlap between these events. Differentiation between arterial and venous anatomy is interpreted by the timing of the image frame where opacified vascular anatomy appears.

To sharpen the contrast between the vascular tree and the non-vascular tissues sufficiently to obtain a diagnostically useful image, the quantity and concentration of the injected chemical vascular contrast agent may need to be high and the x-ray dose also may need to be high. Elevating the contrast dose and/or the x-ray dose increases the signal to noise ratio in the produced angiographic images, but also increases the risk to the subject in several ways.

The injected chemical contrast agent has toxic side effects to kidneys and other internal organs, and therefore it often is necessary to lower the dose of contrast agent to reduce the risk of these toxic side effects. This may produce unsatisfactory images with poor signal to noise ratios which, in turn, may lead to incomplete angiographic studies with inadequately imaged vascular anatomy. Use of an elevated chemical contrast dose may lead to injury to those organs vulnerable to chemical contrast side effects. It may also compel the advancement of the injecting catheter further into the arterial tree so that the injected contrast remains concentrated within the anatomic region of interest. The need to advance the injecting catheter further elevates the risk of complications caused by the catheter injuring ever smaller vessels distal in the vascular tree.

### SUMMARY OF THE INVENTION

Methods of imaging a mammalian host are provided, in which an imaging effective amount of a contrast agent is administered to the host and angiographic data of the host is obtained, where the angiographic data is processed to generate a diagnostically useful image containing a spatiotemporal reconstruction of cardiac frequency angiographic phenomena from the angiographic data, where the cardiac frequency angiographic phenomena is a periodic, physiologically coherent signal with a corresponding cardiac frequency magnitude and a cardiac frequency phase; where the imaging effective amount of the contrast agent is significantly less than the amount required to produce a diagnostically useful image in the absence of extracting the spatiotemporal reconstruction of cardiac frequency angiographic phenomena; and/or where the signal to noise ratio is significantly improved compared to the signal to noise ratio obtained in the absence of extracting the spatiotemporal reconstruction of cardiac frequency angiographic phenomena.

Methods also are provided for reducing the toxicity of imaging a mammalian host, in which an imaging effective amount of a contrast agent is administered to the host and angiographic data of the host is obtained, where the angiographic data is processed to generate a diagnostically useful image containing a spatiotemporal reconstruction of cardiac frequency angiographic phenomena from the angiographic data, where the cardiac frequency angiographic phenomena is a periodic, physiologically coherent signal with a corresponding cardiac frequency magnitude and a cardiac frequency phase; where the effective amount of the contrast agent is significantly less than the amount required to produce a diagnostically useful image in the absence of extracting the spatiotemporal reconstruction of cardiac frequency angiographic phenomena.

In addition, methods are provided for reducing or preventing contrast nephropathy during angiographic imaging of a mammalian host, in which an imaging effective amount of a contrast agent is administered to the host and angiographic data of the host is obtained, where the angiographic data is processed to generate a diagnostically useful image containing a spatiotemporal reconstruction of cardiac frequency angiographic phenomena from the angiographic data, where the cardiac frequency angiographic phenomena is a periodic, physiologically coherent signal with a corresponding cardiac frequency magnitude and a cardiac frequency phase; where the imaging effective amount of the contrast agent is significantly less than the amount required to produce a diagnostically useful image in the absence of extracting the spatiotemporal reconstruction of cardiac frequency angiographic phenomena.

In each of these methods, the imaging may be x-ray imaging. The contrast agent may be an iodine-containing imaging agent, for example, a non-ionic iodine-containing imaging agent, or the contrast agent may be a gadolinium-containing imaging agent.

The imaging effective amount of the contrast agent is at least 25%, at least 50%, or at least 75%, less than the amount required to produce a diagnostically useful image in the absence of extracting the spatiotemporal reconstruction of cardiac frequency angiographic phenomena.

The image may be, for example, an image of; (a) part or all of the heart of the subject, (b) part or all of a kidney of the subject; part or all of the cranium of the subject; and/or part or all of the brain, neck, heart, chest, abdomen, pelvis, legs, feet, arms or hands of the subject.

Also provided are methods of reducing the toxicity of x-ray imaging in a mammalian host, in which an imaging effective amount of a contrast agent is administered to the host and x-ray angiographic data of the host is obtained at faster than cardiac frequency of the host, where the angiographic data is processed to generate a diagnostically useful image containing a spatiotemporal reconstruction of cardiac frequency angiographic phenomena from the angiographic data, where the cardiac frequency angiographic phenomena is a periodic, physiologically coherent signal with a corresponding cardiac frequency magnitude and a cardiac frequency phase; and where the dose of the x-ray required to obtain a diagnostically useful image is significantly less than the amount required to produce a diagnostically useful image in the absence of extracting the spatiotemporal reconstruction of cardiac frequency angiographic phenomena. In these methods the x-ray dosage required to obtain a diagnostically useful image may be at least 25%, at least 50%, or at least 75% less than the amount required to produce a diagnostically useful image in the absence of extracting cardiac frequency magnitude and phase for plurality of pixels.

### BRIEF DESCRIPTION OF THE DRAWINGS

FIG. 1 illustrates the enabling discovery of a method to extract and represent cardiac frequency magnitude and phase, based on a spatiotemporal reconstruction, e.g., using wavelets, in an angiogram that is exploited to offer a process to increase the angiographic informational yield or net signal to noise ratio per pixel.

FIGS. 2A and 2B illustrate how the enabling discovery of the angiographic organization of cardiac frequency magnitude and phase, based on a spatiotemporal reconstruction, e.g., using wavelets, increases the angiographic informational yield while reducing risks and limitations and a corresponding hue brightness legend.

FIGS. 3A and 3B illustrate an angiographic catheter that is navigated further into a vascular tree to obtain an angiogram with a sharper contrast travel profile with a lower dose of contrast and a corresponding cardiac signal profile.

FIGS. 4A and 4B depict a rotational x-ray system that may be used with embodiments of the invention for acquiring angiographic data.

FIG. 5 is a block diagram of a computer system or information processing device that may be used with embodiments of the invention.

FIG. 6 shows a comparison of angiograms obtained with: (1) low contrast agent dose and low x-ray dose, with no wavelet reconstruction; (2) low contrast agent dose and low x-ray dose, with wavelet reconstruction; (3) conventional contrast agent dose and conventional x-ray dose, with no wavelet reconstruction; and (4) conventional contrast agent dose and conventional x-ray dose, with wavelet reconstruction.

### DETAILED DESCRIPTION

Compositions and methods are provided that permit angiographic images and information to be obtained while using lower intravascular contrast dose, lower x-ray dose, and/or less distance navigation of a catheter that injects the angiographic contrast into a vascular tree. The methods use data processing techniques, applied to an angiogram, to generate a spatiotemporal reconstruction, e.g., using wavelets, also referred to as a cardiac space angiogram. See U.S. Pat. No. 10,123,761, the contents of which are hereby incorporated by reference in their entirety. Present techniques exploit the organization of cardiac frequency phase, including coherence, and cardiac frequency from the cardiac space angiogram with regard to reducing x-ray exposure and/or contrast dosage, as well as positioning of the catheter, as described herein. The presence of angiographic coherence increases the net signal in the captured data, and the increase in net signal reduces or eliminates the need for conventional methods of increasing the signal such as increased contrast dose, increased x-ray dose, and further navigation of the injecting catheter. In addition, the coherence between the arterial and venous subsystems of circulation provides a way (other than by the travel timing of a contrast bolus) of providing angiographic contrast. This allows discrimination between arterial and venous angiographic information using a venous injection of contrast, which avoids the risk to the subject of invasion of the arterial system by an injection catheter.

The methods described herein allow reduction of the dose of the intravascular contrast agent used in an angiographic procedure and therefore reduces the risk of toxic side effects caused by the contrast agent in the patient. Most intravascular contrast chemical forms are nephrotoxic, and therefore improved methods that permit use of a lower dose of contrast agent are especially valuable in patients with renal disease, although the skilled artisan will recognize that lowering the dose of contrast agent is advantageous in all patients.

The methods described herein permit the x-ray dose received by a patient during angiography to be reduced, and thereby reduces the risk of harm to the patient from that x-ray radiation. Alternately, for the same total x-ray dose, the methods allow the acquisition of greater imaging information for the same total x-ray dose. Furthermore, angiography health care professionals have some of the highest exposure to x-ray radiation and, accordingly, reducing the x-ray dose has the secondary benefit of sparing incidental x-ray dosing to medical personnel.

Reducing the x-ray dose has the further advantage of reducing the equipment requirements to generate the extra dose and to capture it in an image, and to shield the local environment from the x-ray dose. Reducing the equipment footprint allows equal or greater angiographic imaging information to be obtained from a smaller hardware configuration that draws less electrical power and allows improvements in the field of portable angiography.

Obtaining a diagnostically useful angiographic image often requires advancing the injecting catheter further into the arterial tree so that the injected contrast remains concentrated within the anatomic region of interest. This increases the risk of complications caused by the catheter injuring ever smaller vessels distal in the vascular tree. Increasing the signal-to-noise ratio by exportation of angiographic coherence reduces the procedural risk to the patient by reducing the distance in the vascular tree to which a catheter needs to be advanced for study.

The methods described herein generate an increased signal to noise ratio in an angiographic study by exploiting the organized cardiac frequency magnitude and phase, from a spatiotemporal reconstruction, e.g., using wavelets, within the vascular tree. The methods further exploit the presence of coherence at cardiac frequency between the arterial and venous subsystems of circulation. This means that arteries in an angiogram generally pulse with shared phase, veins generally pulse with shared phase, and these phases do not overlap but instead generally maintain a relatively fixed difference. Exploiting coherence between the arterial and venous components of the circulation allows arterial anatomy to be distinguished from venous anatomy in an angiogram at lower contrast and x-ray doses using criteria other than the travel timing of an injected bolus of chemical contrast agent.

Furthermore, this coherence allows detection of altered patterns of circulation, such as the disruption or occlusion of an artery or of a vein. Such an injury alters the coherence relationship between the arterial and venous sides of a vascular bed, providing biomarkers for the disruption of the vascular tree.

### Spatiotemporal Reconstructions Of Cardiac Frequency Phenomena

Method for extracting vascular anatomy and physiology information are provided by analyzing the patterns of cardiac frequency magnitude, phase, and coherence in a spatiotemporal reconstruction of cardiac frequency phenomena extracted from an angiogram obtained at faster than cardiac frequency. The spatiotemporal reconstructions of cardiac frequency phenomena are described in detail in U.S. Pat. No. 10,123,761, the contents of which are hereby incorporated by reference in their entirety.

The term “cardiac space angiogram” as used herein refers to the totality of the product of a spatiotemporal reconstruction of cardiac frequency phenomena as described by the '761 patent. The cardiac space angiogram includes not only the spatiotemporal reconstructions of cardiac frequency phenomena as generated by a computer program, but also the angiogram that the computer program operates upon. Accordingly, the cardiac space angiogram includes all of the information of a conventional angiogram plus the additional information contained in the spatiotemporal reconstruction of the cardiac frequency phenomena. Advantageously, the method described by the '761 patent is applied in a computer program to generate a cardiac space angiogram, however the skilled artisan will recognize that other methods of reconstructing the spatiotemporal cardiac frequency activity may be used.

A cardiac space angiogram is based on angiographic images acquired at faster than cardiac rate, in compliance with the sampling theorem of Nyqvist, Kotelnikov, and Shannon, as known in the art. This method can resolve single vascular pulse waves, as distinguished from cardiac gated methods where one cardiac cycle is interpolated from many.

As described above, the signal at cardiac frequency in an angiogram is exploited to increase the sensitivity of angiographic imaging to arterial anatomy and to venous anatomy, allowing identification of altered and pathological patterns of circulation such as vessel occlusions and other blood flow states at lower x-ray doses and at lower intravascular contrast doses. Additionally, it allows the separation of arterial from venous anatomy without navigating and injecting a catheter into the distal arterial tree. The coherence at cardiac frequency among circulatory sub-systems may be exploited to allow the anatomic identification of arterial anatomy and venous anatomy at lower x-ray doses and at lower intravascular contrast doses.

In carrying out the methods described herein, the angiographic data are recorded using a digital detector device, such as those commercially available as part of scanning devices available from manufacturers such as Philips and Siemens. The digital data are then imported into a computer memory. After the import into computer memory of an angiogram (in the absence of motion alias), the spatiotemporal reconstruction of cardiac frequency angiographic phenomena may be obtained by the following steps:
the angiographic data consisting of n by m pixels by q frames data is imported into computer memory and reformatted with the processor in memory to give an n by m array of time signals each q samples long;
a complex valued wavelet transform is applied by the processor to each pixel-wise time signal, giving an n by m array of wavelet transforms;
the pixel-wise wavelet transforms are filtered for cardiac frequency by the processor.

This is done by setting to zero all wavelet coefficients that do not correspond to cardiac wavelet scale (in the field of wavelets this term corresponds to the concept of cardiac frequency);
the pixel-wise wavelet transforms data are inverse wavelet transformed by the processor into time domain and reformatted in computer memory into q frames of n by m pixels. Each data element (voxel) in this three dimensional grid is a complex valued number;
each frame can be rendered as an image with a brightness hue color model to represent the complex datum in each pixel by the processor;
cardiac frequency magnitude is represented as brightness and phase as hue; and
the q images may be rendered as motion cine by the processor or they may be stored as a video file format by the processor.

Any suitable transform, operable on complex numbers that retain time indexing after transformation into the frequency domain, and capable of extracting the spatiotemporal reconstruction of cardiac frequency angiographic phenomena is contemplated for use with the present techniques.

### Contrast Agents

The methods described herein provide methods of greatly reducing the dose of contrast agent required to obtain a diagnostically useful angiogram. A “diagnostically useful” angiogram is one that provides the person reading the angiogram (such as a radiologist) with data of a quality sufficient to provide meaningful clinical information and/or to allow treatment decisions to be made. Although a reduction in dose of contrast agent is generally desirable for all subjects undergoing angiography, contrast nephropathy is particularly problematic for patients with impaired kidney function or who are otherwise renally vulnerable. See, generally, Mavromatis, “The Imperative of Reducing Contrast Dose in Percutaneous Coronary Intervention,” Cardiovascular Interventions 7:1294-1296 (2014). Accordingly, such patients particularly benefit from using the instant methods to reduce or prevent contrast nephropathy during angiographic imaging.

The main type of contrast agent used in angiography is the family of iodinated contrast agents, which can be ionic or, advantageously, non-ionic iodinated contrast agents. Such agents are well known in the art and include: iohexol (Omnipaque™, GE Healthcare); iopromide (Ultravist™, Bayer Healthcare); iodixanol (Visipaque™, GE Healthcare); ioxaglate (Hexabrix™, Mallinckrodt Imaging); iothalamate (Cysto-Conray II™, Mallinckrodt Imaging); and iopamidol (Isovue™, Bracco Imaging). See also Lusic and Grinstaff, “X-Ray Computed Tomography Contrast Agents,” Chem Rev. 13:1641-66 (2013). Other agents include gadolinium-based agents. See Ose et al., “‘Gadolinium’ as an Alternative to Iodinated Contrast Media for X-Ray Angiography in Patients With Severe Allergy,” Circ J. 2005; 69:507-509 (2005).

The dosages for such contrast agents vary depending on the nature of the agent, the physical characteristics of the patient/subject, and the nature of the angiographic procedure. In general however, the contrast agent should improve the visualization of the target tissue by increasing the absolute CT attenuation difference between the target tissue and surrounding tissue and fluids by a factor of ≈2x. The imaging media should contain a high mol% of the x-ray attenuating atom per agent (molecule, macromolecule, or particle) in order to reduce the volume used and concentrations needed for imaging. Also, the tissue retention-time of the contrast agent should be sufficiently long for completion of a CT scan and scheduling the instrument time in the diagnostic setting (e.g., 2-4 h). Moreover, the contrast agent advantageously should: (a) localize or target the tissue of interest and possess favorable biodistribution and pharmacokinetic profiles; (b) be readily soluble or form stable suspensions at aqueous physiological conditions (appropriate pH and osmolality) with low viscosity; (c) be non-toxic; and (d) be cleared from the body in a reasonably short amount of time, usually within several hours (<24 h).

Even if a contrast agent meets these criteria, it is generally desirable to reduce the dosage used for imaging, and this is particularly the case for patients with reduced kidney function or who have an allergic or other adverse reaction to the agent. The methods described herein allow the use of a dose of contrast agent that is significantly less than the dosage that would otherwise be required to provide diagnostically useful imaging information. In this context of contrast agent dosage, a dose is significantly less if it less than 75%, less than 50%, less than 40%, less than 30%, less than 25%, less than 20%, less than 15%, less than 10%, less than 5% or less than 3% of the dose that would otherwise be required to produce a diagnostically useful angiogram.

### X-Ray Dosage

The x-ray dosage required to generate a diagnostically useful image in an angiogram also varies depending on physical characteristics of the patient/subject and the nature of the angiographic procedure. Methods of calculating x-ray dosages are well known in the art. The ionizing nature of x-ray radiation means that it is always desirable to minimize the exposure of the subject (and medical staff associated with an angiography procedure) to x-rays as much as possible while still producing a useful visualization of the target tissue. In this context of x-ray dosage, an x-ray dose is significantly less if it less than 75%, less than 50%, less than 40%, less than 30%, less than 25%, less than 20%, less than 15%, less than 10%, less than 5% or less than 3% of the x-ray dose that would otherwise be required to produce a diagnostically useful angiogram.

### Signal to Noise Ratio

The signal to noise ratio of an angiogram depends, inter alia, on both the dosage of the contrast agent, and the dose of the x-ray. The instant methods allow for a significant increase in the signal to noise ratio for a given dose of contrast agent and/or x-ray dosage. In the context of the instant methods, a significant increase or improvement of the signal to noise ratio is one that permits the dosage of either the contrast agent and/or the x-ray to be less than 75%, less than 50%, less than 40%, less than 30%, less than 25%, less than 20%, less than 15%, less than 10%, less than 5% or less than 3% of the dose that would otherwise be required to produce a diagnostically useful angiogram

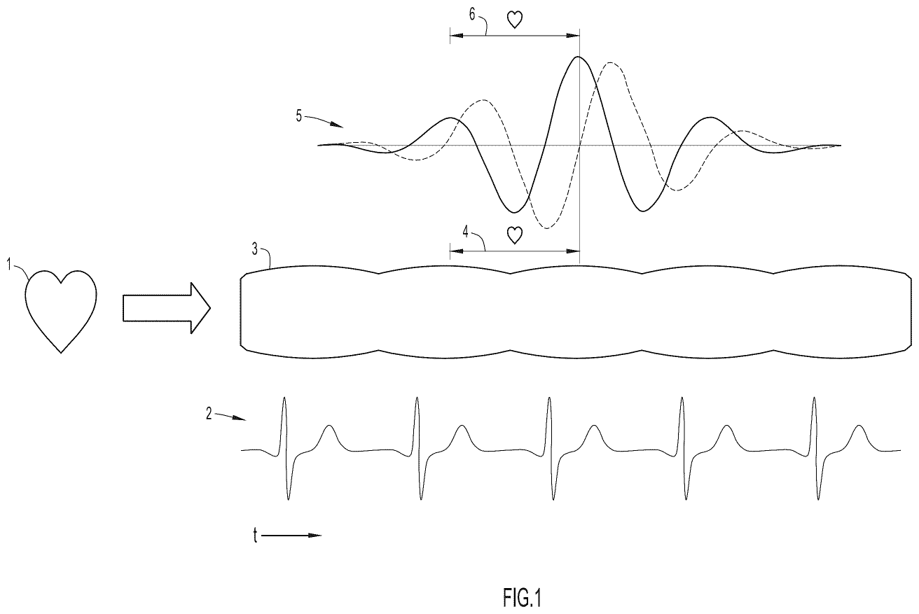

The arrangement in FIG. 1 provides a schematic that shows how the methods described herein provide increased signal to noise ratio compared to conventional angiography. In FIG. 1, a heart 1 sends blood to the body as a sequence of arterial stroke volumes. The work of the heart generates a cardiac signal. An example of this is the electrocardiogram 2. An artery 3 has contrast variation produced by the traveling arterial stroke volumes. The time between the arterial stroke volumes is the cardiac period 4, and is also the time between cardiac cycles in the electrocardiogram 2. A mother wavelet 5 function is created in a computer program with a wavelet cardiac scale 6 that matches the cardiac period 4 of the cardiac signal. Advantageously, the Gabor wavelet family is selected for mother wavelet 5. The Gabor mother wavelet is complex-valued, and has a real component (solid black) and an imaginary component (dashed) in the mother wavelet 5. The use of a complex-valued mother wavelet facilitates the extraction and representation of cardiac frequency magnitude and phase, based on a spatiotemporal reconstruction, e.g., using wavelets. The arrangement of FIG. 1 is repeated pixel-by-pixel across the image frames of an angiographic study to produce a cardiac space angiogram, which is a cine spatiotemporal representation of cardiac frequency phenomena in the angiogram. For clarity, the arrangement of FIG. 1 does not include other reconstruction steps that are executed in the computer program such as those that mitigate motion alias in balance with frequency alias in the reconstructed result.

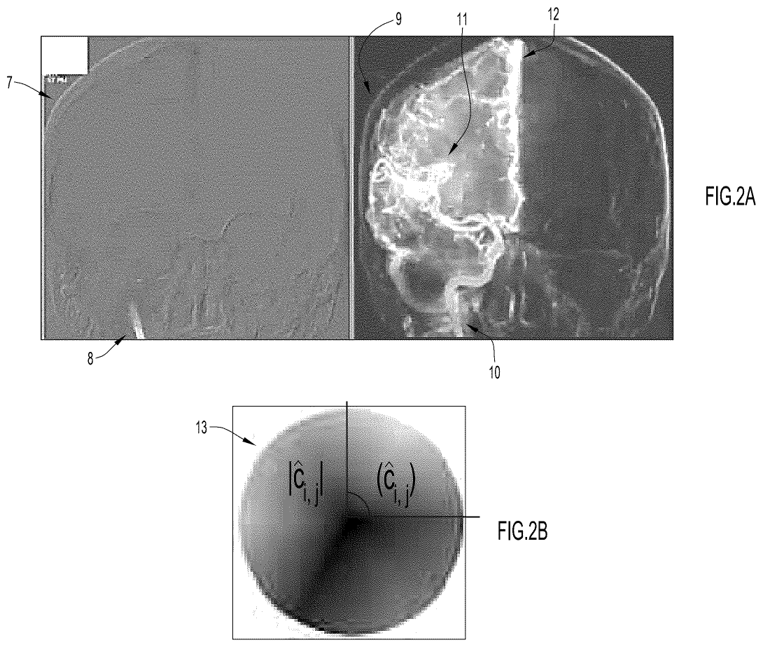

The arrangement in FIG. 2A shows an example angiogram and illustrates how the discovery may be exploited to lower intravascular contrast dose, enable the manufacture of lower footprint angiography hardware, and other desirable outcomes. FIG. 2A shows an image frame from a low dose conventional angiogram of the right internal carotid artery of the brain 7 and an image frame of a cardiac space angiogram corresponding to the same frame 9. At a low contrast and x-ray dose, right internal carotid artery 8 of the arterial subsystem is partially opacified but other circulatory subsystems are not opacified. In the image frame of a cardiac space angiogram corresponding to the same frame 9, several circulatory subsystems can be observed, including right internal carotid artery cardiac frequency phenomena 10, intervening vasculature between the right internal carotid artery cardiac frequency and the venous subsystem 11, and venous sub-system 12. Every pixel in the image represents a complex-valued datum. Each complex valued datum c may be rendered with a brightness-hue color model where cardiac frequency magnitude is rendered as brightness and phase as hue according to the legend brightness-hue legend 13. The anatomic demonstration of a relatively complete arterial subsystem, intervening subsystem, and venous subsystem in an image frame of a cardiac space angiogram corresponding to the same frame 9 reflects the additional informational yield from exploiting the informational yield of cardiac frequency angiographic phenomena. This extra informational yield serves as a biomarker for reducing the risk of angiography, for making it more efficient, and for facilitating the manufacture of lighter footprint angiography hardware.

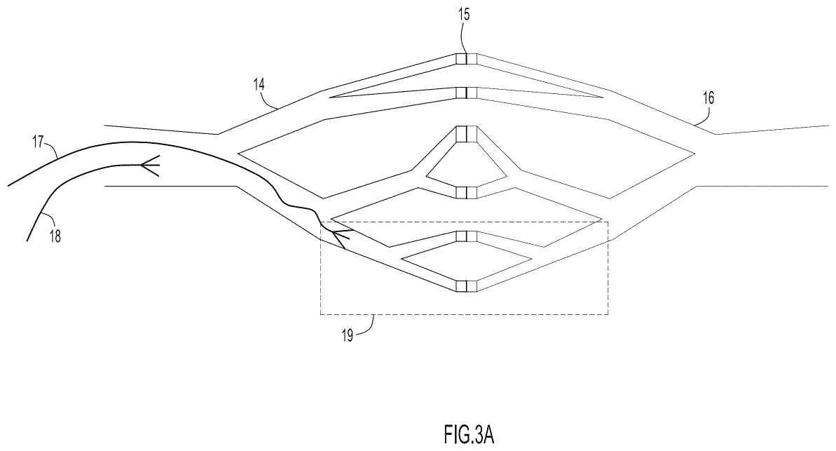

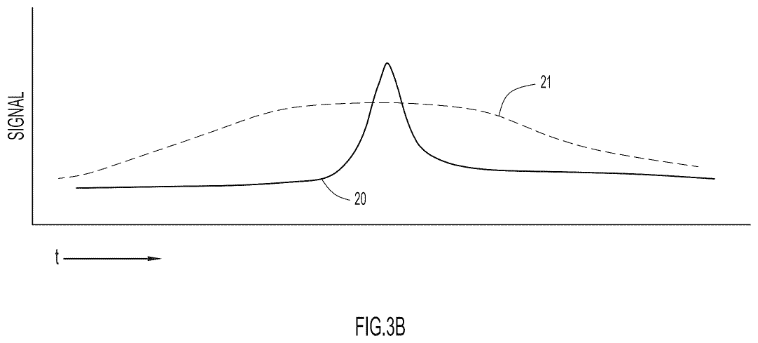

The arrangement in FIG. 3A depicts improvements in the benefit to risk profile of an angiography process offered by exploiting angiographic coherence. A vascular tree (19) contains an arterial subsystem 14, a capillary subsystem of the vascular tree 15, and venous subsystem of the vascular tree 16. There may be an injection catheter navigated distal into the vascular tree 17 (solid black) to inject a relatively smaller volume of intravascular chemical contrast agent and opacify a smaller portion of the vascular tree. There may be an injection catheter navigated proximal into the vascular tree 18 (dashed black) where a larger quantity of intravascular chemical contrast agent is injected to opacify a larger portion of the vascular tree. The injection catheter navigated distal into the vascular tree 17 produces a result where the travel allows greater discrimination of the arterial from venous subsystems. The injection catheter navigated proximal into the vascular tree 18 produces a result that reduces discrimination of the arterial from venous subsystems. The coherence at cardiac frequency between the arterial and venous subsystems provides a way to distinguish the arterial from venous subsystems other than by the travel timing of the passing contrast bolus. This allows those subsystems to be distinguished by a less sharp bolus injection and by a more safely placed proximal catheter, or even an injection from the venous system. Using the cardiac phenomenon described herein allows for methods where a distance from a catheter tip (black lines) to the capillary subsystem (15) of the vascular tree is significantly increased compared to a system that does not use the cardiac phenomenon. Exploiting the cardiac frequency signal in the injected contrast provides an increased signal to noise profile of all aspects of the angiogram, thereby allowing both intravascular chemical contrast dose and/or x-ray dose to be reduced. FIG. 3B shows a corresponding cardiac signal profile, showing magnitude 20 and phase 21.

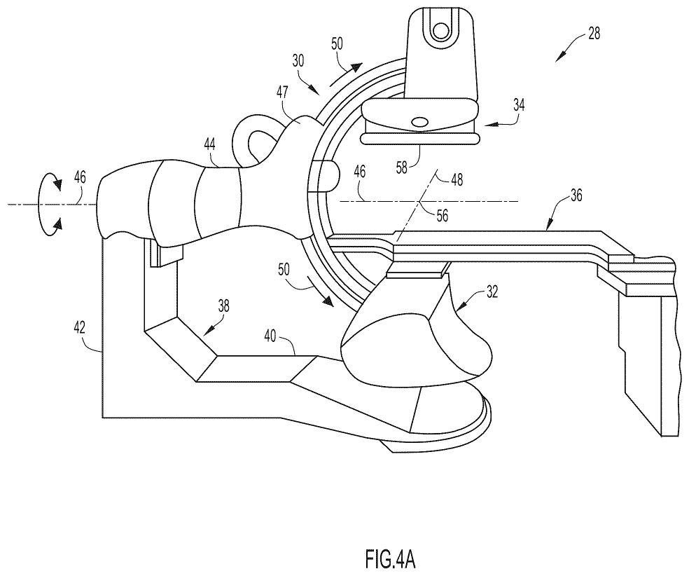

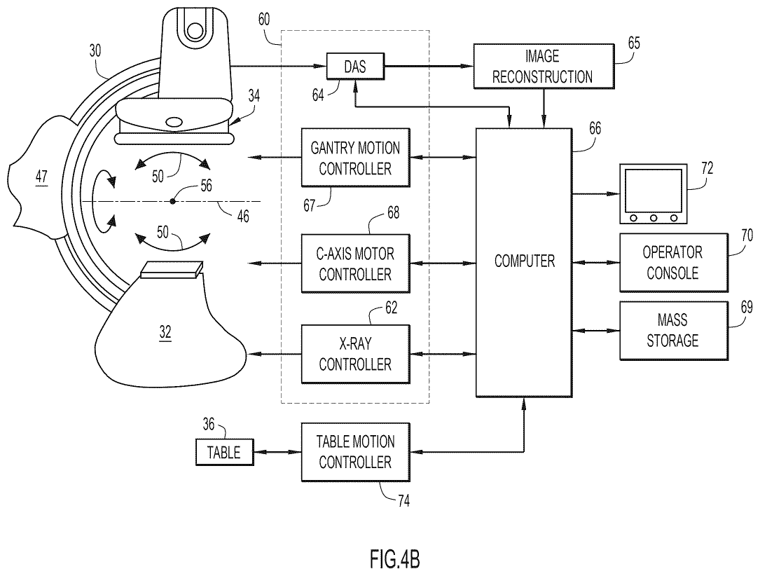

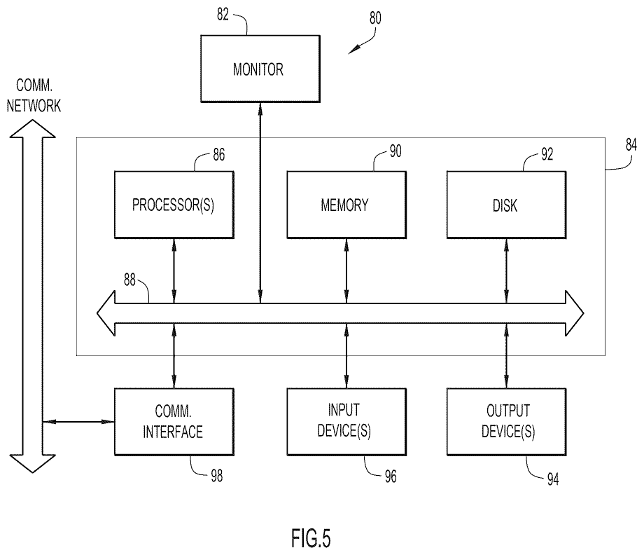

Referring to FIGS. 4A and 4B, a rotational x-ray system 28 is illustrated that may be used to obtain an angiogram at a faster than cardiac rate, such as via fluoroscopic angiography. As previously described, in acquiring an angiograph, a chemical contrast agent is injected into the patient and the contrast opacifies the vessels and allows their projections to be captured by the x-ray system as a two-dimensional image. However, the embodiments provided herein are not limited to two-dimensions, but may be applied to images acquired in three or more dimensions. A sequence of these two dimensional projection images is acquired that comprises an angiographic study-with the angiographic image frames acquired at faster than cardiac frequency to allow spatiotemporal reconstruction of the cardiac frequency phenomena into a cardiac space angiogram.

As shown in FIG. 4A, the rotational x-ray system 28 is characterized by a gantry having a C-arm 30 which carries an x-ray source assembly 32 on one of its ends and an x-ray detector array assembly 34 at its other end. The gantry enables the x-ray source 32 and detector 34 to be oriented in different positions and angles around a patient disposed on a table 36, while enabling a physician access to the patient. The gantry includes a pedestal 38 which has a horizontal leg 40 that extends beneath the table 36 and a vertical leg 42 that extends upward at the end of the horizontal leg 40 that is spaced from of the table 36. A support arm 44 is rotatably fastened to the upper end of vertical leg 42 for rotation about a horizontal pivot axis 46.

The pivot axis 46 is aligned with the centerline of the table 36, and the arm 44 extends radially outward from the pivot axis 46 to support a C-arm drive assembly 47 on its outer end. The C-arm 30 is slidably fastened to the drive assembly 47 and is coupled to a drive motor (not shown) which slides the C-arm 30 to revolve it about a C-axis 48 as indicated by arrows 50. The pivot axis 46 and C-axis 48 intersect each other, at an isocenter 56 located above the table 36, and are perpendicular to each other.

The x-ray source assembly 32 is mounted to one end of the C-arm 30 and the detector array assembly 34 is mounted to its other end. The x-ray source 32 emits a beam of x-rays which are directed at the detector array 34. Both assemblies 32 and 34 extend radially inward to the pivot axis 46 such that the center ray of this beam passes through the system isocenter 56. The center ray of the beam thus can be rotated about the system isocenter around either the pivot axis 46 or the C-axis 48, or both, during the acquisition of x-ray attenuation data from a subject placed on the table 36.

The x-ray source assembly 32 contains an x-ray source which emits a beam of x-rays when energized. The center ray passes through the system isocenter 56 and impinges on a two-dimensional flat panel digital detector 58 housed in the detector assembly 34. The detector 58 may be, for example, a 2048×2048 element two-dimensional array of detector elements. Each element produces an electrical signal that represents the intensity of an impinging x-ray and hence the attenuation of the x-ray as it passes through the patient. During a scan, the x-ray source assembly 32 and detector array assembly 34 are rotated about the system isocenter 56 to acquire x-ray attenuation projection data from different angles. The detector array is able to acquire 50 projections, or views, per second which is the limiting factor that determines how many views can be acquired for a prescribed scan path and speed.

Referring to FIG. 4B, the rotation of the assemblies 32 and 34 and the operation of the x-ray source are governed by a control mechanism 60 of the x-ray system. The control mechanism 60 includes an x-ray controller 62 that provides power and timing signals to the x-ray source 32. A data acquisition system (DAS) 64 in the control mechanism 60 samples data from detector elements and passes the data to an image reconstructor 65. The image reconstructor 65 receives digitized x-ray data from the DAS 64 and performs high speed image reconstruction according to the methods of the present disclosure. The reconstructed image is applied as an input to a computer 66 which stores the image in a mass storage device 69 or processes the image further.

The control mechanism 60 also includes gantry motor controller 67 and a C-axis motor controller 68. In response to motion commands from the computer 66, the motor controllers 67 and 68 provide power to motors in the x-ray system that produce the rotations about respective pivot axis 46 and C-axis 48. The computer 66 also receives commands and scanning parameters from an operator via console 70 that has a keyboard and other manually operable controls. An associated display 72 allows the operator to observe the reconstructed image and other data from the computer 66. The operator supplied commands are used by the computer 66 under the direction of stored programs to provide control signals and information to the DAS 64, the x-ray controller 62 and the motor controllers 67 and 68. In addition, computer 66 operates a table motor controller 74 which controls the motorized table 36 to position the patient with respect to the system isocenter 56.

Referring now to FIG. 5, a block diagram of a computer system or information processing device 80 is illustrated that may be used with rotational x-ray system 28 of FIGS. 4A and 4B for the extraction of cardiac frequency phenomena and the exploitation of cardiac frequency phenomena as biomarkers of properties of circulatory anatomy and physiology, according to an embodiment of the present invention.

FIG. 5 is merely illustrative of a general-purpose computer system 80 programmed according to techniques within this disclosure or a specific information processing device for an embodiment incorporating an invention whose teachings may be presented herein and does not limit the scope of the invention. One of ordinary skill in the art will recognize that other variations, modifications, and alternatives to computer system 80 may be used.

In one embodiment, computer system 80 includes monitor 82, computer 84 (which includes processor(s) 86, bus subsystem 88, memory subsystem 90, and disk subsystem 92), user output devices 94, user input devices 96, and communications interface 98. Monitor 82 can include hardware and/or software elements configured to generate visual representations or displays of information. Some examples of monitor 82 may include familiar display devices, such as a television monitor, a cathode ray tube (CRT), a liquid crystal display (LCD), or the like. In some embodiments, monitor 82 may provide an input interface, such as incorporating touch screen technologies.

Computer 84 can include familiar computer components, such one or more central processing units (CPUs), memories or storage devices, graphics processing units (GPUs), communication systems, interface cards, or the like. As shown in FIG. 2, computer 84 may include one or more processor(s) 86 that communicate with a number of peripheral devices via bus subsystem 88. Processor(s) 86 may include commercially available central processing units or the like. Bus subsystem 88 can include mechanisms for letting the various components and subsystems of computer 84 communicate with each other as intended. Although bus subsystem 88 is shown schematically as a single bus, alternative embodiments of the bus subsystem may utilize multiple bus subsystems. Peripheral devices that communicate with processor(s) 86 may include memory subsystem 90, disk subsystem 92, user output devices 94, user input devices 96, communications interface 98, or the like.

Memory subsystem 90 and disk subsystem 92 are examples of physical storage media configured to store data. Memory subsystem 90 may include a number of memories including random access memory (RAM) for volatile storage of program code, instructions, and data during program execution and read only memory (ROM) in which fixed program code, instructions, and data are stored. Disk subsystem 92 may include a number of file storage systems providing persistent (non-volatile) storage for programs and data. Other types of physical storage media include floppy disks, removable hard disks, optical storage media such as CD-ROMS, DVDs and bar codes, semiconductor memories such as flash memories, read-only-memories (ROMS), battery-backed volatile memories, networked storage devices, or the like.

Memory subsystem 90 and disk subsystem 92 may be configured to store programming and data constructs that provide functionality or features of techniques discussed herein. Software code modules and/or processor instructions that when executed by processor(s) 86 implement or otherwise provide the functionality may be stored in memory subsystem 90 and disk subsystem 92.

User input devices 94 can include hardware and/or software elements configured to receive input from a user for processing by components of computer system 80. User input devices can include all possible types of devices and mechanisms for inputting information to computer system 84. These may include a keyboard, a keypad, a touch screen, a touch interface incorporated into a display, audio input devices such as microphones and voice recognition systems, and other types of input devices. In various embodiments, user input devices 94 can be embodied as a computer mouse, a trackball, a track pad, a joystick, a wireless remote, a drawing tablet, a voice command system, an eye tracking system, or the like. In some embodiments, user input devices 94 are configured to allow a user to select or otherwise interact with objects, icons, text, or the like that may appear on monitor 82 via a command, motions, or gestures, such as a click of a button or the like.

User output devices 96 can include hardware and/or software elements configured to output information to a user from components of computer system 80. User output devices can include all possible types of devices and mechanisms for outputting information from computer 84. These may include a display (e.g., monitor 82), a printer, a touch or force-feedback device, audio output devices, or the like.

Communications interface 98 can include hardware and/or software elements configured to provide unidirectional or bidirectional communication with other devices. For example, communications interface 98 may provide an interface between computer 84 and other communication networks and devices, such as via an internet connection.

FIG. 5 is representative of a computer system capable of embodying the present invention. It will be readily apparent to one of ordinary skill in the art that many other hardware and software configurations are suitable for use with the present invention. For example, the computer may be a desktop, portable, rack-mounted or tablet configuration. Additionally, the computer may be a series of networked computers. In still other embodiments, the techniques described above may be implemented upon a chip or an auxiliary processing board.

The usefulness of vascular coherence in wavelet angiography is demonstrated in the Example below, in which greatly reduced dosage of both contrast agent and x-ray radiation was used while providing improved diagnostic information.

### EXAMPLE

Two human angiograms were performed in immediate succession in anteroposterior (AP) projection of the right vertebral artery. Since iodinated contrast and x-ray have injurious properties, a so-called “puff” angiogram (preparatory angiogram) was obtained using 10% of the dose of the iodinated contrast agent and 1% of the x-ray dose conventionally used for a diagnostic angiogram.

For the “puff” injection the chemical agent used was iopamidol (Isovue), at a dose of 1 ml of a formulation of 3 mg/ml, which provides a dose of 3 mg iopamidol for the injection. The x-ray dose area product was 1.968 Gray m². The “dose area product” is a measure of the absorbed dose per kilogram multiplied by the area irradiated. The x-ray dose is obtained from the image series DICOM metadata.

For the regular (“full dose”) right vertebral artery injection, the injected contrast dose was also iopamidol, but using 10 ml of the same formulation of 3 mg/ml, providing a dose of 30 mg iopamidol for the injection. The x-ray dose product was 156.876 Gray m².

The puff injection therefore used 10% of the dose of the iopamidol chemical contrast dose and 1.3% of the x-ray dose.

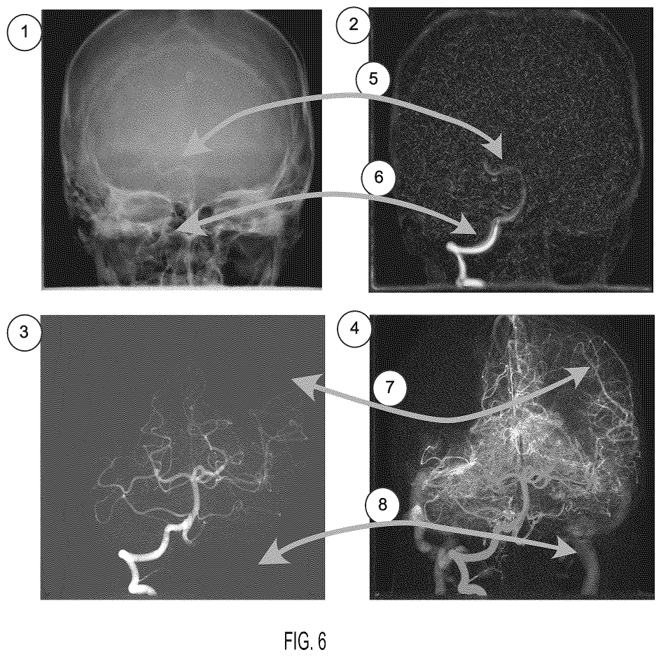

The results obtained are shown in the top row (1 and 2) of FIG. 6. A puff angiogram typically is used to verify the correct position of a subject's body between the x-ray emitted tube and the x-ray detector, and to verify the integrity of the iodinated contrast injection catheters. A full angiogram of the same subject, using the conventional doses of contrast reagent and x-ray radiation is shown in the bottom row (3 and 4) of FIG. 6.

In the top left puff angiogram (1) the data are shown without wavelet reconstruction. The left arrow head of the double-headed arrow (5) shows a trace of contrast in a cerebral blood vessel. The finding of this vessel means that the subject is ready and appropriately situated for delivery of the full iodinated contrast dose through the injection catheter and the application of full x-ray dose (3)(also shown without wavelet reconstruction).

The angiogram (1) obtained without wavelet reconstruction can be compared to the angiogram (2) obtained with wavelet reconstruction. One vessel indicated by the double-headed arrow (5) can be seen with and without wavelet reconstruction-the left arrowhead shows the image without wavelet reconstruction, while the right arrowhead shows the superior image with wavelet reconstruction. The bones of the skull base in the conventional puff angiogram (1) block sufficient passage of x-rays to view the passage of the vessel across the skull base. Hence, a vessel indicated by the left arrow head of double-headed arrow (6) is not clearly seen.

By contrast, the puff angiogram with wavelet reconstruction shows the passage of the vessel through the skull base (right arrow head of double-headed arrow (6)). This is because those image pixels are varying in intensity at cardiac frequency, even though in a given image frame they do not have enough x-ray attenuation contrast to be seen in the conventional puff image (1).

The bottom row (3) and (4) of FIG. 6 shows the angiogram at conventional iodinated contrast and x-ray doses (3) and its wavelet reconstruction (4). The wavelet angiogram (4) shows arteries (double-headed arrow (7)) and veins (double-headed arrow (8)) that are not visible in the conventional angiogram (3).

Other objects, features and advantages of the methods described herein will be apparent from the detailed description. It should be understood, however, that the detailed descriptions provided herein are given by way of illustration only, since various changes and modifications within the spirit and scope of the invention will become apparent to those skilled in the art.

What is claimed is:

1. A method of imaging a mammalian host, comprising:
administering an imaging effective amount of a contrast agent to the host and obtaining angiographic data of the host,
wherein the angiographic data is processed to generate a diagnostically useful image comprising a spatiotemporal reconstruction of cardiac frequency angiographic phenomena from the angiographic data, wherein the cardiac frequency angiographic phenomena is a periodic, physiologically coherent signal with a corresponding cardiac frequency magnitude and a cardiac frequency phase;
wherein the imaging effective amount of said contrast agent is significantly less than the amount required to produce a diagnostically useful image in the absence of extracting the spatiotemporal reconstruction of cardiac frequency angiographic phenomena; and/or
wherein the signal to noise ratio is significantly improved compared to the signal to noise ratio obtained in the absence of extracting the spatiotemporal reconstruction of cardiac frequency angiographic phenomena.

2. A method of reducing the toxicity of imaging a mammalian host, comprising:
administering an effective amount of a contrast agent to the host and obtaining angiographic data of the host,
wherein the angiographic data is processed to generate a diagnostically useful image comprising a spatiotemporal reconstruction of cardiac frequency angiographic phenomena from the angiographic data, wherein the cardiac frequency angiographic phenomena is a periodic, physiologically coherent signal with a corresponding cardiac frequency magnitude and a cardiac frequency phase;
wherein the effective amount of said contrast agent is significantly less than the amount required to produce a diagnostically useful image in the absence of extracting the spatiotemporal reconstruction of cardiac frequency angiographic phenomena.

3. A method of reducing or preventing contrast nephropathy during angiographic imaging of a mammalian host, comprising:
administering an imaging effective amount of a contrast agent to the host and obtaining angiographic data of the host,
wherein the angiographic data is processed to generate a diagnostically useful image comprising a spatiotemporal reconstruction of cardiac frequency angiographic phenomena from the angiographic data, wherein the cardiac frequency angiographic phenomena is a periodic, physiologically coherent signal with a corresponding cardiac frequency magnitude and a cardiac frequency phase;
wherein the imaging effective amount of said contrast agent is significantly less than the amount required to produce a diagnostically useful image in the absence of extracting the spatiotemporal reconstruction of cardiac frequency angiographic phenomena.

4. The method according to claim 1 wherein the imaging is x-ray imaging.

5. The method according to claim 1, wherein the contrast agent is an iodine-containing imaging agent.

6. The method according to claim 5, wherein the contrast agent is a non-ionic iodine-containing imaging agent.

7. The method according to claim 4, wherein the contrast agent is a gadolinium-containing imaging agent.

8. The method according to claim 1, wherein the imaging effective amount of said contrast agent is at least 25% less than the amount required to produce a diagnostically useful image in the absence of extracting the spatiotemporal reconstruction of cardiac frequency angiographic phenomena.

9. The method according to claim 8, wherein the imaging effective amount of said contrast agent is at least 50% less than the amount required to produce a diagnostically useful image in the absence of extracting the spatiotemporal reconstruction of cardiac frequency angiographic phenomena.

10. The method according to claim 8, wherein the imaging effective amount of said contrast agent is at least 75% less than the amount required to produce a diagnostically useful image in the absence of extracting the spatiotemporal reconstruction of cardiac frequency angiographic phenomena.

11. The method according to claim 1, wherein the image comprises an image of the heart of the subject.

12. The method according to claim 1, where the image comprises an image of part or all of the heart of the subject.

13. The method according to claim 1, where the image comprises an image of part or all of a kidney of the subject.

14. The method according to claim 1, where the image comprises an image of part or all of the cranium of the subject.

15. The method according to claim 1, where the image comprises an image of part or all of the brain, neck, heart, chest, abdomen, pelvis, legs, feet, arms or hands of the subject.

16. A method of reducing the toxicity of x-ray imaging in a mammalian host, comprising:
administering an effective amount of a contrast agent to the host and obtaining x-ray angiographic data at faster than cardiac frequency of the host,
wherein the angiographic data is processed to generate a diagnostically useful image comprising a spatiotemporal reconstruction of cardiac frequency angiographic phenomena from the angiographic data, wherein the cardiac frequency angiographic phenomena is a periodic, physiologically coherent signal with a corresponding cardiac frequency magnitude and a cardiac frequency phase;
wherein the dose of the x-ray required to obtain a diagnostically useful image is significantly less than the amount required to produce a diagnostically useful image in the absence of extracting the spatiotemporal reconstruction of cardiac frequency angiographic phenomena.

17. The method according to claim 16, wherein the x-ray dosage required to obtain a diagnostically useful image is at least 25% less, at least 50% less, or at least 75% less than the amount required to produce a diagnostically useful image in the absence of extracting cardiac frequency magnitude and phase for plurality of pixels.
